(19) United States
(12) Patent Application Publication
(10) Pub. No.: US 2008/0200889 A1
(43) Pub. Date: Aug. 21, 2008

(54) Diaper Incontinent Pad Combo DIP-C and Dualie Diaper

(76) Inventor: Walter Louis Sanders, Rowlett, TX (US)

Correspondence Address: WILSON DANIEL SWAYZE, JR., 3804 CLEARWATER CT., PLANO, TX, 75025, US

(21) Appl. No.: 11/675,256

(22) Filed: Feb. 15, 2007

(51) Int. Cl. A61F 13/15

(52) U.S. Cl. 604/358; 604/385.01

(57) ABSTRACT

An incontinence pad for user includes a first moisture impenetrable exterior liner on a first side of the incontinence pad, a second moisture impenetrable exterior liner on the first side of the incontinence pad, a first flexible portion to connect the first moisture impenetrable exterior liner to the second moisture impenetrable exterior liner, a moisture punishable interior liner to form a continuous sealed edge with the first moisture impenetrable exterior liner and the second moisture impenetrable exterior liner. The incontinence pad includes a weakened area to convert the incontinence pad to a diaper. A diaper assembly for a user includes a first disposable diaper, and a second disposable diaper detachably connected to the first disposable diaper. The second disposable diaper remains connected to the first disposable diaper as the first disposable diaper is being used by the user.

### BACKGROUND OF THE INVENTION

1. Field of the Invention

This invention relates generally to diapers and incontinent pads and more particularly to a pair of detachedly connected diapers and a incontinent pad which can be converted to the diaper.

2. General Background

Parents now have the option of using a variety of diapers for their child, ranging from the conventional cloth diaper washed at home or provided by a laundry service, disposable diapers, or a combination of these types in an all-in-one diaper. Arguments are made on all sides regarding the health and sanitation issues, the energy and environmental cost, as well as the economics involved in using each type. However, case studies have clearly indicated that it all comes down to a personal choice based on the life style and economic advantages of the parent.

An issue focuses on the comfort of the baby with respect to the diaper applied. It is essential that the diaper be snug to insure retention and prevent leakage but not so tight as to be uncomfortable or hamper blood circulation in the child's legs due to elastic leg openings and not so tight as to create high humidity within the diaper.

Some studies even suggest the use of two diapers per changing to help insure dryness. Therefore, elastic covers or diapers with hook and loop fasteners that can cut and abrade the child and the addition of disposable multi-layers of absorbent material is clearly not the answer.

Further, hook and loop fasteners generally only allow for waist adjustment and do nothing for the leg openings. Therefore, elastic is often used around the leg openings. This arrangement assumes that the waist and leg size are relative which is certainly not the case.

Obviously the key issues are to insure that the baby stays as dry as possible for as long as possible with the least bulk possible. The baby's comfort should be paramount and not dependent on convenience for the diaper changer.

Furthermore, when traveling away from home, carrying spare diapers is a major concern. An additional concern especially when the caregiver only has a small pocketbook is where to place the spare diaper.

Furthermore, incontinence pads are generally larger than diapers and are used frequently for bed patients. However, when these patients get up, they frequently require a diaper. In some cases, the incontinence pads are clean but thrown out unused. This can cause a large and wasteful expense.

### SUMMARY

An incontinence pad for user includes a first moisture impenetrable exterior liner on a first side of the incontinence pad, a second moisture impenetrable exterior liner on the first side of the incontinence pad, a first flexible portion to connect the first moisture impenetrable exterior liner to the second moisture impenetrable exterior liner, a moisture punishable interior liner to form a continuous sealed edge with the first moisture impenetrable exterior liner and the second moisture impenetrable exterior liner. The incontinence pad includes a weakened area to convert the incontinence pad to a diaper.

The continuous seal is maintained after the incontinence pad is converted to the diaper.

The incontinence pad includes a first centrally located absorbent porous fibrous core of the first thickness, and the incontinence pad includes a second absorbent porous fibrous core of the second thickness. The first thickness is greater than the second thickness.

A diaper assembly for a user includes a first disposable diaper, and a second disposable diaper detachably connected to the first disposable diaper. The second disposable diaper remains connected to the first disposable diaper as the first disposable diaper is being used by the user.

The diaper assembly includes a first fastening device to fasten to the user and the diaper assembly includes a second fastening device to detachedly connect the first disposable diaper and the second disposable diaper.

The second fastening device includes Velcro and detachable adhesive.

### BRIEF DESCRIPTION of the DRAWINGS

The invention may be understood by reference to the following description taken in conjunction with the accompanying drawings, in which, like reference numerals identify like elements, and in which:

FIG. 1 illustrates a cross-sectional view of the disposable garment of the present invention;

FIG. 2 illustrates a cross-sectional side view of the disposable garment of the present invention;

FIG. 3 illustrates a cross-sectional back view of the disposable garment of the present invention;

FIG. 4 illustrates a cross-sectional front view of the disposable garment of the present invention after a section has been removed;

FIG. 5 illustrates a cross-sectional view of the detachedly connected diapers of the present invention; and

FIG. 6 illustrates a cross-sectional side view of the detachedly connected diapers of the present invention.

### DETAILED DESCRIPTION

### Diaper Incontinent Pad Combo-DIP-C

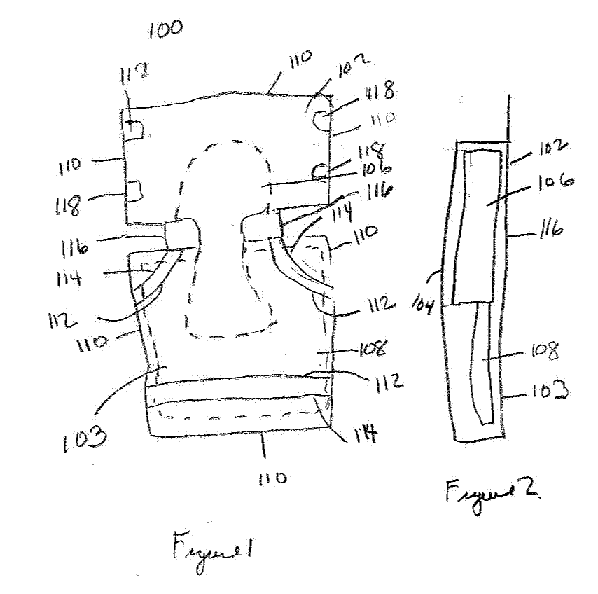

FIG. 1 illustrates the disposable garment 100 such as a incontinence pad of the present invention and the disposable garment 100 includes a first moisture impermeable exterior liner 102 positioned on a first side of the disposable garment 100 connected to a first flexible portion 116 which is connected to a second moisture impermeable exterior liner 103. The first moisture impermeable exterior liner 102 and the second moisture impermeable exterior liner 103 are shown as substantially rectangular in shape but other shapes could be used. The disposable garment 100 additionally includes a first centrally located absorbent porous fibrous core 106 of a first thickness and a second absorbent porous fibrous core 108 of a second thickness. The first thickness may be thicker than the second thickness. The first centrally located absorbent porous fibrous core 106 may be substantially a ‘light bulb’ shape or could be another suitable shape.

The first moisture impermeable exterior liner 102 may include a fastening device 118 such as velco, a releasable adhesive, buttons or any other suitable device.

The first moisture impermeable exterior liner 102, the second moisture impermeable exterior liner 103 and the first flexible portion 106 form a continuous sealed edge 110 with the moisture permeable interior liner 104 which is positioned on the second side of the incontinence pad to help prevent any liquid from escaping.

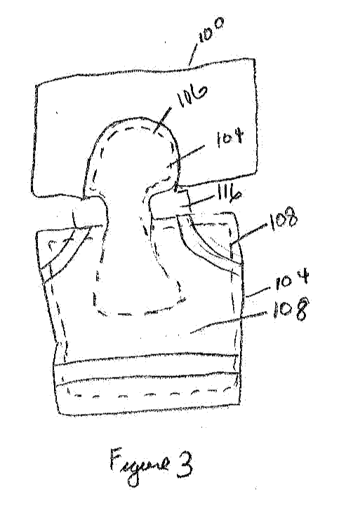

FIG. 2 illustrates a side view of the first moisture impermeable exterior liner 102, the second moisture impermeable exterior liner 103, the first flexible portion 106 attached to the moisture permeable interior liner 104 to hold the first centrally located absorbent, porous fibrous core 106 and the second absorbent porous fibrous core 108.

In addition, the disposable garment includes an interior sealed edge 112 and a weakened area 114 to remove a portion of the second moisture impermeable exterior liner 103 in order to convert the incontinent garment such as bedpan to a diaper.

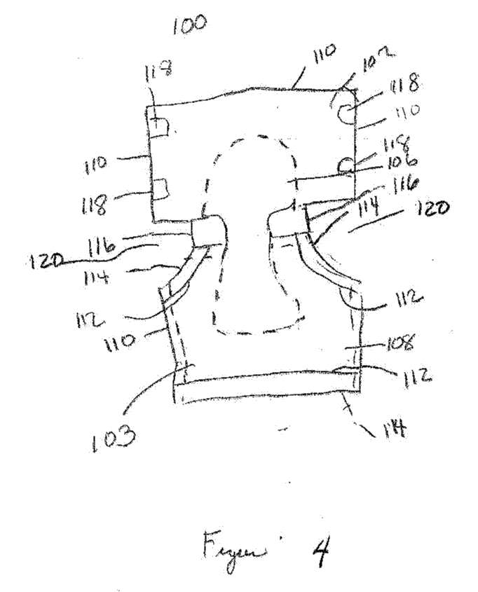

FIG. 3 illustrates the interior side of the disposable garment 100 showing the moisture permeable interior liner 104.

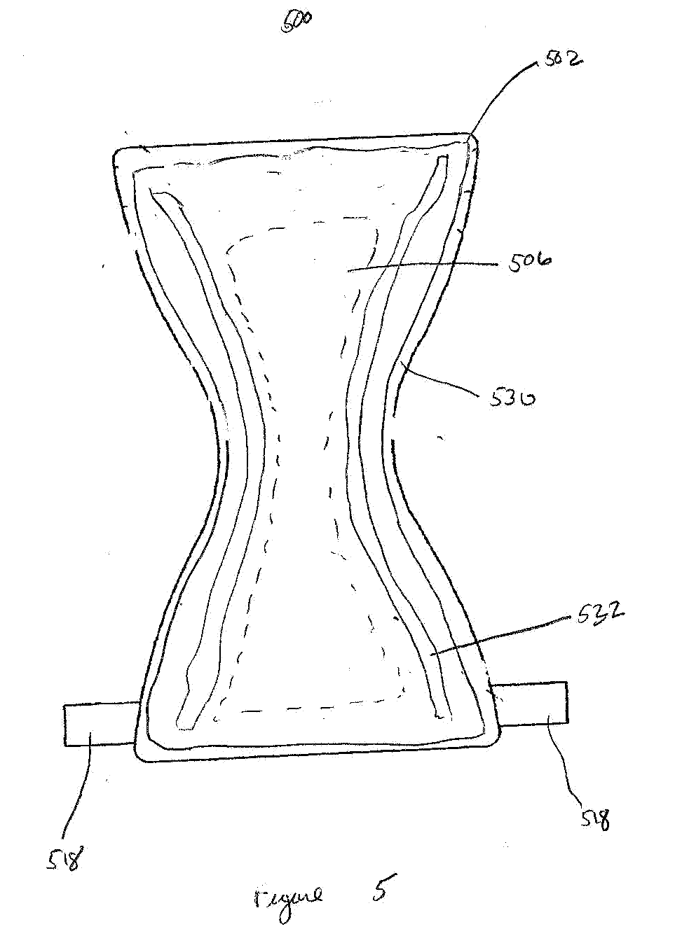

FIG. 4 illustrates the effects of removing the portions of the second moisture impermeable exterior liner 103 and the opposing moisture permeable interior liner 104 defined by the weakened area 114. It should be noted that the interior sealed edge 112 maintains the seal of the disposable garment. The legs of the user can be inserted into the area defined by the narrow portion, and the approximate upper and lower portions which can be pulled against the user and attached by the fastening device 118 to provide a diaper for the user.

### Dualie Diaper

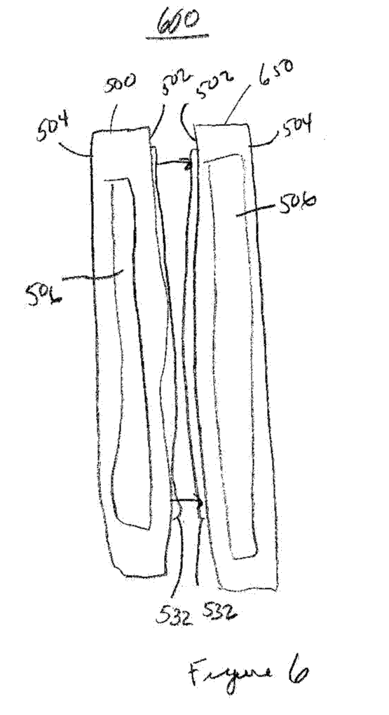

FIG. 5 illustrates another embodiment of the present invention which shows a first disposable diaper 500 which can be detachably connected to a second disposable diaper 650. FIG. 5 illustrates the first disposable diaper 500 which includes a centrally located absorbent porous fibrous core section 506 which can retain disposable waste from a user. The first disposable diaper 600 includes a gathering material 530 stretched to form a periphery elastic edge portion of the first disposable diaper 650. The first disposable diaper 500 includes first fastening device 518 which may be Velcro, detachable adhesive, buttons or other suitable devices to attached to the opposing end of the first disposable diaper 500. The first disposable diaper 500 includes a second fastening device 532 positioned along the longitudinal periphery of the first disposable diaper 500 to fasten to an opposing second fastening device 532 of a second disposable diaper 650 as shown in FIG. 6. FIG. 6 illustrates a diaper assembly 600 including the first disposable diaper 500 and the second disposable diaper 650 The second fastening devices 532 of the first disposable diaper 500 and the second disposable diaper 650 are tracks so that the first disposable diaper 500 may be detachedly attached to the second disposable diaper 650. The first disposable diaper 500 and the second disposable diaper 650 may be detachedly connected while the first disposable diaper 500 is being worn by the user. After the user soils the first disposable diaper 500, the caretaker separates the second disposable diaper 650 from the first disposable diaper 500, and disposes the first disposable diaper 500. Subsequently, the user wears the second disposable diaper 650. The second fastening devices 532 may be Velcro, detachably adhesive, buttons, belts or any other suitable device. The first and second disposable diapers which are detachably connected free the user or caregiver from the necessity of separately carrying another diaper. The user of the diaper carries his/her replacement.

The gathered material 530 may be an elastic-like stretchable material to give the diaper a comfortable fit and shape and to prevent leakage.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. An incontinence pad for user, comprising:
a first moisture impenetrable exterior liner on a first side of the incontinence pad;
a second moisture impenetrable exterior liner on the first side of the incontinence pad;
a first flexible portion to connect the first moisture impenetrable exterior liner to the second moisture impenetrable exterior liner;
a moisture punishable interior liner to form a continuous sealed edge with the first moisture impenetrable exterior liner and the second moisture impenetrable exterior liner;
wherein the incontinence pad includes a weakened area to convert the incontinence pad to a diaper.

2. An incontinence pad for user as in claim 1, wherein the continuous seal is maintained after the incontinence pad is converted to the diaper.

3. An incontinence pad for user as in claim 1, wherein the incontinence pad includes a first centrally located absorbent porous fibrous core of the first thickness.

4. An incontinence pad for user as in claim 3, wherein the incontinence pad includes a second absorbent porous fibrous core of the second thickness.

5. An incontinence pad for user as in claim 4, wherein the first thickness is greater than the second thickness.

6. A diaper assembly for a user, comprising:
a first disposable diaper;
a second disposable diaper detachably connected to the first disposable diaper;
wherein said second disposable diaper remained connected to the first disposable diaper as the first disposable diaper is being used by the user.

7. A diaper assembly for a user as in claim 6, wherein the diaper assembly includes a first fastening device to fasten to the user.

8. A diaper assembly for a user as in claim 7, wherein the diaper assembly includes a second fastening device to detachedly connect the first disposable diaper and the second disposable diaper.

9. A diaper assembly for a user as in claim 8, wherein the second fastening device includes Velcro.

10. A diaper assembly for a user as in claim 8, wherein the second fastening device includes detachable adhesive.
